(19) United States
(12) Patent Application Publication
(10) Pub. No.: US 2003/0104372 A1
(43) Pub. Date: Jun. 5, 2003

(54) Allele specific primer extension

(75) Inventors: Afshin Ahmadian, Stockholm (SE); Joakim Lundeberg, Stockholm (SE); Pal Nyren, Skarpnack (SE)

Correspondence Address: DORSEY & WHITNEY LLP, 250 Park Avenue, New YOrk, NY, 10177, US

(73) Assignee: PYROSEQUENCING AB.

(21) Appl. No.: 09/791,190

(22) Filed: Feb. 23, 2001

Related U.S. Application Data
(63) Continuation-in-part of application No. 09/331,517, filed on Jul. 23, 1999, now Pat. No. 6,258,568.
Application No. 09/331,517 is a 371 of international application No. PCT/GB97/03518, filed on Dec. 22, 1997.

(30) Foreign Application Priority Data
Dec. 23, 1996 (GB) 9626815.6

(51) Int. Cl. C12Q 1/68; C12P 19/34

(52) U.S. Cl. 435/6; 435/91.2

(57) ABSTRACT

The present invention provides methods of allele-specific primer extension useful for detecting mutations and genetic variations.

### CROSS REFERENCE TO RELATED APPLICATION

This application is a continuation-in-part of U.S. application Ser. No. 09/331,517, which is a 371 of PCT/GB97/03518 filed Dec. 22, 1997, the disclosure of which is incorporated herein by reference.

### BACKGROUND OF THE INVENTION

Analysis of single nucleotide polymorphisms (SNPs) is useful in applications including mapping, linkage studies and pharmacogenomics. Consequently, a number of different techniques have been proposed to scan these sequence variations in a high-throughput fashion. Many of these methods originate from hybridization techniques to discriminate between allelic variants. High-throughput hybridization of allele-specific oligonucleotides can be performed on microarray chips (Wang et al. (1998) Science 280:1077), microarray gels (Yershov et al. (1996) Proc. Natl. Acad. Sci. USA 93:4913), or by using allele-specific probes (molecular beacons) in the polymerase chain reaction (PCR) (Tyagi et al. (1998) Nat. Biotechnol. 16:49). Other technologies which have been shown to be useful for SNP genotyping are minisequencing (Pastinen et al. (1997) Genome Res. 7:606), mass spectrometry (Laken et al. (1998) Nat. Biotechnol. 16:1352) and pyrosequencing (Ahmadian et al. (2000) Anal. Biochem. 280:103), the latter relying on incorporation of nucleotides by DNA polymerase with an enzymatic cascade converting the released pyrophosphate (PPi) into detectable light.

The use of pairs of allele-specific primers with alternative bases at the 3′ end has been used to identify single base variations. Higgins et al. (1997) Biotechniques 23:710; Newton et al. (1989) Lancet 2:1481; Goergen et al. (1994) J. Med. Virol. 43:97; and Newton et al. (1989) Nucleic Acids Res. 17:2503. This method exploits the difference in primer extension efficiency by a DNA polymerase of a matched over a mismatched primer 3′-end. Generally, a sample is divided into two extension reaction mixtures that contain the same reagents except for the primers, which differ at the 3′-end. The alternating primer is designed to match one allele perfectly but mismatch the other allele at the 3′-end. Because the polymerase differs in extension efficiency for matched versus mismatched 3′-ends, the allele-specific extension reaction thus provides information on the presence or absence of one allele.

The foregoing method of identifying single base variations using allele-specific primers with varying 3′-ends suffers from certain deficiencies. In particular, certain mismatches, such as G:T and C:A, are poorly discriminated by the DNA polymerase, leading to false positive signals (Day et al. (1999) Nucleic Acids Res. 27:1810). In these cases, DNA polymerase extends the mismatched primer-templates in the presence of nucleotides although, as the present inventors have shown, with slower reaction kinetics as compared to extension of the matched configuration. However, the kinetic difference is usually not distinguishable in end point analysis, such as allele-specific PCR.

The present invention solves the deficiencies of the prior art by providing a method of allele-specific extension that allows accurate discrimination between matched and mismatched configurations. The present methods are useful for high throughput SNP analysis.

### SUMMARY OF THE INVENTION

The presence invention provides methods of allele-specific primer extension useful for detecting mutations and genomic variations. In one embodiment, a nucleotide degrading enzyme, preferably apyrase, is included in the allele-specific primer reaction. In this method, nucleotides are degraded before extension in reactions having slow kinetics due to mismatches, but not in reactions having fast kinetics due to matches of primer and allelic target.

In another embodiment, the primers for the allele-specific primer extension reactions are designed such that the 3′-end base is complementary to the target, the penultimate (3′-1 end) base is allele-specific, and the base two positions from the 3′-end (3′-2 end) is the same as (i.e. non-complementary to) the target. When one mismatch is present (2 bases from the 3-end) the primer target duplex is stable and extension occurs. However, when two mismatches are present (at the 3′-1 and 3′-2 positions), duplex stability is disrupted and no detectable extension occurs.

### BRIEF DESCRIPTION OF THE DRAWINGS

FIG. 1 depicts the results of allele-specific extension for the three variants of the single nucleotide polymorphisms codon 72 (p53) and wiaf 1764. The SNP status, which was determined separately for each sample, and the extension primers are shown at the top of the Figure. FIG. 1a shows the raw data obtained using the luminometric assay without apyrase (top panel) and with apyrase (lower panel). The arrows point out the signal of pyrophosphate (0.02 μM) which was added to the reaction mixture prior to the nucleotide addition in all samples to serve as a positive control as well as for peak calibration. FIG. 1B shows the extension results of these samples using fluorescently labeled nucleotides spotted on a glass slide, with apyrase (lower panel) and without apyrase (top panel).

FIG. 2 is a schematic depiction of a method of apyrase mediated allele specific extension using barcodes as tags on the 5′-ends of allele-specific primers.

FIG. 3 is a schematic depiction of a method of allele-specific extension in solution followed by hybridization to a DNA microarray.

FIG. 4 is a schematic depiction of an apyrase mediated allele specific reaction in which Taq Man probes are used to measure primer extension.

FIG. 5 depicts raw data of an apyrase mediated allele specific reaction utilizing a microarray format.

FIG. 6 depicts bioluminometric analysis of a SNP by double allele-specific primer extension on double stranded DNA.

FIG. 7 depicts allele specific extension using a primer having an introduced mismatch.

### DETAILED DESCRIPTION OF THE INVENTION

The present invention relates to methods for detecting bases in a nucleic acid target. The present methods are useful for detecting mutations and genomic variations, and particularly single nucleotide polymorphisms (SNPs), and may be used with single stranded or double stranded DNA targets.

In one embodiment, the present invention provides a method of detecting a base at a predetermined position in a DNA molecule. The method utilizes DNA polymerase catalyzed primer extension. Pairs of primers are designed that are specific for (i.e. complementary to) an allele of interest, but that differ at the 3′-terminus, which position corresponds to the polymorphic nucleotide. In one primer, the 3′-terminus is complementary to the non-mutated nucleotide; in the other primer, the 3′-terminus is complementary to the mutated nucleotide. The primers are used in separate extension reactions of the same sample. Depending upon whether the mutation is present, the 3′-terminus of the primer will be a match or mismatch for the target. The DNA polymerase discriminates between a match and mismatch, and exhibits faster reaction kinetics when the 3′-terminus of the primer matches the template. Thus measurement of the difference in primer extension efficiency by the DNA polymerase of a matched over a mismatched 3′-terminus allows the determination of a non-mutated versus mutated target sequence. In accordance with the present invention it has been found that the addition of a nucleotide degrading enzyme in the extension reaction minimizes the extension of mismatched primer configurations by removal of nucleotides before incorporation but allows extension when reaction kinetics are fast, i.e. in a matched configuration. The present invention thus reduces or eliminates false positive results seen in prior art methods.

In accordance with the present invention, samples may be prepared, primers synthesized, and primer-extension reactions conducted by methods known in the art, and disclosed for example by Higgins et al. (1997) Biotechniques 23:710; Newton et al. (1989) Lancet 2:1481; Goergen et al. (1994) J. Med. Virol. 43:97; and Newton et al. (1989) Nucleic Acids Res. 17:2503, the disclosures of which are incorporated by reference. SNP sites may be amplified prior to analysis, for example by PCR, including nested and multiplex PCR. SNP containing templates may be immobilized.

The nucleotide degrading enzyme is preferably added after primer hybridization and preferably with the extension reaction mixture. For PCR reactions, a thermostable nucleotide degrading enzyme is used.

In a preferred embodiment of the present method, the nucleotide degrading enzyme is apyrase, which is commercially available, for example from Sigma Chemical Co., St. Louis, Mo. USA. Those of ordinary skill in the art can determine a suitable amount of apyrase or other nucleotide degrading enzyme to degrade unincorporated nucleotides in extension reactions having slow kinetics due to a mismatch at the 3′-terminus of the primer. For example, a typical 50 μl extension reaction mixture may contain for 1 to 15 mU, and preferably from 5 to 10 mU, of apyrase.

In the present invention, primer extension can be measured, and thus the ratio of extension using the primer having a matched 3′-terminus and extension using the primer having an unmatched 3′-terminus determined, by methods known in the art. If two primers are used in each reaction, PCR products can be generated and measured. Other methods of measuring primer extension include mass spectroscopy (Higgins et al. (1997) Biotechniques 23:710), luminometric assays, in which incorporation of nucleotides is monitored in real-time using an enzymatic cascade, (Nyren et al. (1997) Anal. Biochem. 244:367), fluorescent assays using dye-labeled nucleotides, or pyrosequencing as described by Ronaghi et al. (1998) Science 281:363, and Ahmadian et al. (2000) Anal. Biochem. 280:103.

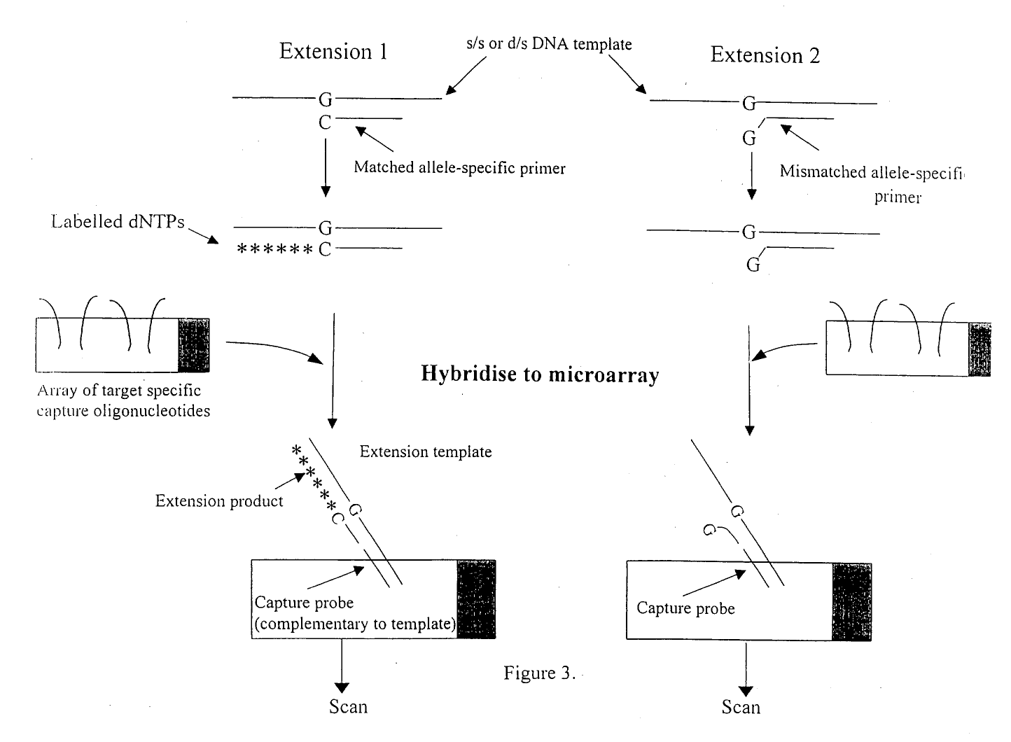

The present method may be performed in a solid phase microarray format, for example on a chip, whereby samples are extended in solution followed by hybridization to a microarray. (FIG. 3). Allele-specific primer extension on microarrays is known in the art and described for example by Pastinen et al. (2000) Genome Research 10:1031. The method may also be performed a liquid phase assay using barcodes as tags on the 5′-ends of allele-specific primers as described by Fan et al. (2000) Genome Res. 10:853. By using barcodes, a liquid-phase multiplex apyrase mediated allele specific extension of a set of SNPs may be performed in a single tube (if the barcodes on the matched and mismatched primers are different) or in two tubes (if the barcodes on the matched and mismatched primers are identical). After primer extension, the extension products can be hybridized to barcodes on the chip. A modular probe as described by O'Meara et al. (1998) Anal. Biochem. 255:195 and O'Meara et al. (1998) J. Clin. Microbiol. 36:2454 may be utilized to improve the hybridization efficiency. The modular probe hybridizes to its complementary segment on the immobilized oligonucleotide and improves the hybridization of a barcode that is immediately downstream.

The present method may also be performed using “Taq Man” probes, which are probes labeled with a donor-acceptor dye pair that functions via fluorescence resonance transfer energy (FRET) as described by Livak et al. (1995) PCR Methods Appl. 4:357. When Taq Man probes are hybridized to a target, the fluorescence of the 5′-donor fluorophore is quenched by the 3′-acceptor. When the hybridized probe is degraded, the 5′-donor dye dissociates from the 3′-quencher, leading to an increase in donor fluorescence.

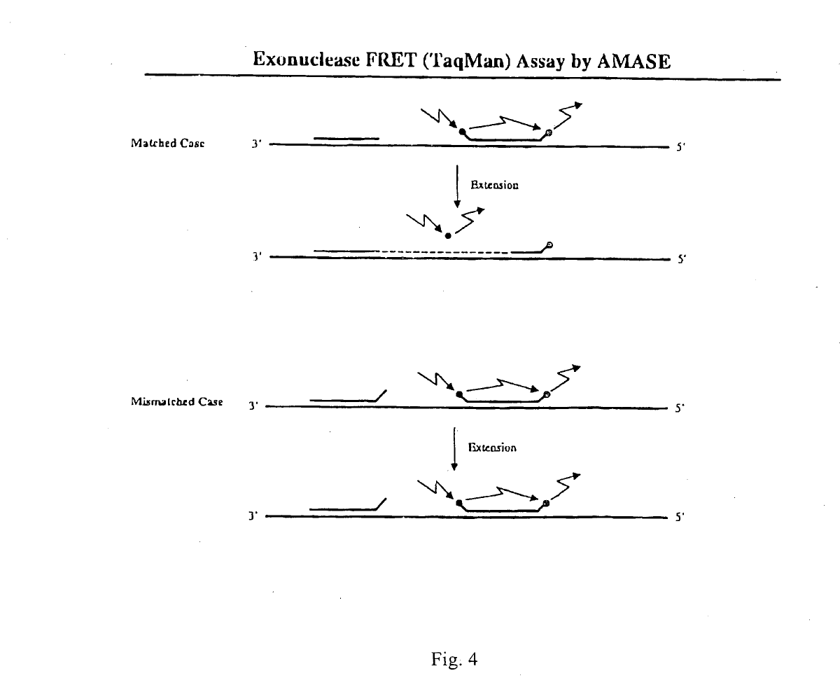

After PCR amplification, a Taq Man probe is used which hybridizes 15 to 20 bases downstream of a 3′-end allele-specific primer. Apyrase-mediated allele specific extension is then performed. In the case of a matched primer-template, DNA polymerase extends the primer and degrades the Taq Man probe by its 5′-nuclease activity, thus leading to increased donor fluorescence. In the mismatched case, apyrase degrades the nucleotides and the fluorescence level remains unchanged. Such an assay is depicted in FIG. 4.

Extension products may also be distinguished by mass difference or by the use of double-stranded-specific intercalating dyes.

In another embodiment of the present invention, allele specific primer extension with a nucleotide degrading enzyme is performed using a double stranded DNA template. If double stranded DNA is generated by PCR, the excess of primers, nucleotides and other reagents must first be removed by methods known in the art such as treatment with alkaline phosphatase and exonuclease I. Two pairs of allele-specific primers are used. One pair is complementary to the forward strand and one to the reverse strand. The primer pairs differ in their 3′-ends as described above. Allele-specific extension is performed on both strands of the double-stranded DNA.

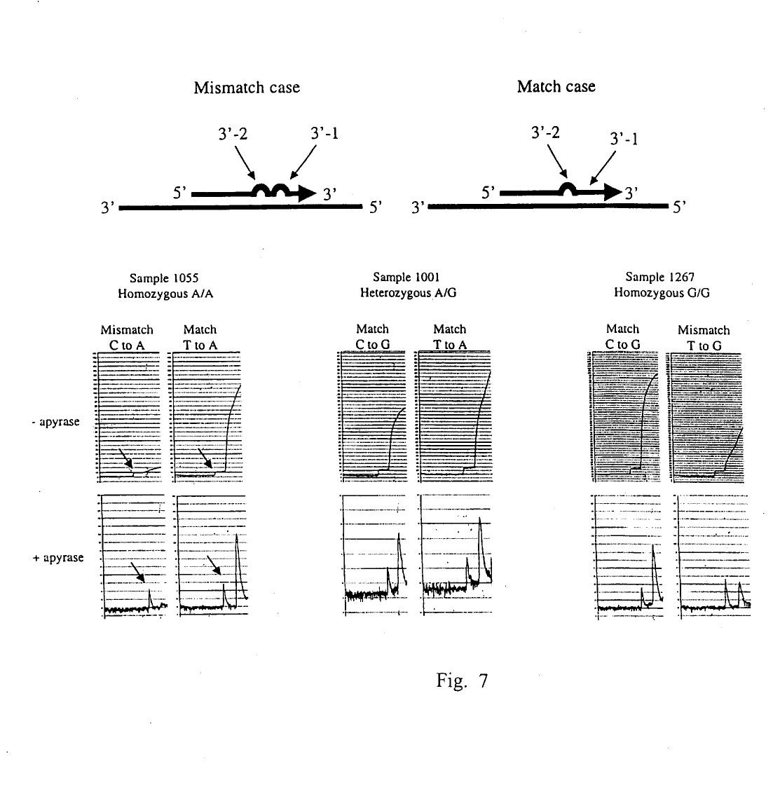

In another embodiment of the present invention, allele specific extension is performed using a pair of primers in which the 3′-end base is complementary to the target, the penultimate (3′-1 end) base is allele-specific (mutated or nonmutated) and the base two positions from the 3′-end (3′-2 end) is the same as (i.e. non-complementary to) the target. When the allele-specific base in the primer matches the target template, i.e. one mismatch is present (2 bases from the 3′-end), the primer-target duplex is stable and extension occurs. When the allele-specific base does not match the target, i.e. two mismatches are present (at the 3′-1 and 3′-2 positions), duplex stability is disrupted and no detectable extension occurs. The assay may be performed in all the embodiments as described above, in the presence or absence of a nucleotide degrading enzyme. An example of the method is depicted in FIG. 7.

All references cited herein are incorporated herein in their entirety.

The following non-limiting examples serve to further illustrate the present invention.

### EXAMPLE 1

### Apyrase Mediated Allele Specific Extension

Experimental Protocol

Samples, PCR and Single Strand Preparation

Human genomic DNA was extracted from twenty-four unrelated individuals. Two duplex PCRs were performed to amplify four SNPs. The SNPs were wiaf1764 (A/C) on chromosome 9q, codon 72 (C/G) on the p53 gene (Ahmadian et al. (2000) Anal. Biochem. 280:103) nucleotide position 677 (C/T) on the MTHFR gene (Goyette et al. (1998) Mamm. Genome 9:652, erratum in (1999) Mamm. Genome 10:204) and nucleotide position 196 (A/G) on the GPIIIa gene (Newman et al. (1989) J. Clin. Invst. 83:1778). The outer duplex PCR for wiaf 1764 and p53 gene (94° C. 1 min, 50° C. 40s and 72° C. 2 min for 35 cycles) was followed by specific inner PCRs (94° C. 1 min, 50° C. 40s and 72° C. 1 min for 35 cycles) generating ˜80 bp fragments for each SNP (Table 1). The outer duplex PCR condition for MTHFR and GPIIIa genes was 95° C. 30s, 60° C. 1 min and 72° C. 1 min. This was followed by individual inner PCRs (95° C. 1 min, 66° C. 50 sec and 72° C. 2 min) (Table 1). The outer and inner amplification mixtures comprised of 10 mM Tris-HCl (pH 8.3), 2 mM MgCl₂, 50 mM KCl, 0.1% (v/v) Tween 20, 0.2 mM dNTPs, 0.1 μM of each primer and 1 unit of AmpliTaq DNA polymerase (Perkin-Elmer, Norwalk, Conn., USA) in a total volume of 50 μl. Five microliters of total human DNA (1 ng/μl) were used as outer PCR template. In the inner PCR, one of the primers in the respective set was biotinylated at the 5′-end to allow immobilization. 40 μl biotinylated inner PCR products were immobilized onto streptavidin-coated super paramagnetic beads (Dynabeads M280; Dynal, Oslo, Norway). Single-stranded DNA was obtained by incubating the immobilized PCR product in 12 μl 0.1 M NaOH for 5 min. The immobilized strand was then used for hybridization to extension primers (Table 1).

Apyrase Mediated Allele-specific Extension Using a Bioluminometric Assay

The immobilized strand was resuspended in 32 μl H₂O and 4 μl annealing buffer (100 mM Tris-acetate pH 7.75, 20 mM Mg-acetate). The single stranded DNA was divided into two parallel reactions in a microtiter-plate (18 μl/well) and 0.1 μM (final concentration) of primers were added to the single stranded templates in a total volume of 20 μl. Allele discrimination between the allelic variants was investigated by the use of Klenow DNA polymerase using two separate SNP primers that only differed in the 3′-end position (Table 1). Hybridization was performed by incubation at 72° C. for 5 min and then cooling to room temperature. The content of each well was then further divided into two separate reactions for comparison of extension analysis with and without apyrase. Extension and real-time luminometric monitoring was performed at 25° C. in a Luc96 pyrosequencer instrument (Pyrosequencing, Uppsala, Sweden). An extension reaction mixture was added to the single stranded DNA (10 μl) with annealed primer (the substrate) to a final volume of 50 μl. The extension reaction mixture contained 10 U exonuclease-deficient (exo-) Klenow DNA polymerase (Amersham Pharmacia Biotech, Uppsala, Sweden), 4 μg purified luciferase/ml (BioThema, Dalarö, Sweden), 15 mU recombinant ATP sulfurylase, 0.1 M Tris-acetate (pH 7.75), 0.5 mM EDTA, 5 mM Mg-acetate, 0.1% (w/v) bovine serum albumin (BioThema), 1 mM dithiothreitol, 10 μM adenosine 5′-phosphosulfate (APS), 0.4 mg polyvinylpyrrolidone/ml (360 000), 100 μg D-luciferin/ml (BioThema) and 8 mU of apyrase (Sigma Chemical Co., St. Louis, Mo., USA) when applicable. Prior to nucleotide addition and measuring of emitted light, pyrophosphate (PPi) was added to the reaction mixture (0.02 μM). The PPi served as a positive control for the reaction mixture as well as peak calibration. All the four nucleotides (Amersham Pharmacia Biotech, Uppsala, Sweden) were mixed and were dispensed to the extension mixture (1.4 μM, final concentration). The emitted light was detected in real-time.

Apyrase Mediated Allele-specific Extension Using Fluorescent Labeled Nucleotide

The single stranded templates were prepared using magnetic beads as outlined above. The immobilized strand was resuspended in 100 μl H₂O and 12 μl annealing buffer (100 mM Tris-acetate pH 7.75, 20 mM Mg-acetate). The single stranded DNA was divided into two parallel reactions in a microtiter-plate (56 μl/well) and 0.1 μM (final concentration) of primers (Table 1) were added to the single stranded templates in a total volume of 60 μl. Hybridization was performed by incubation at 72° C. for 5 min and then cooling to room temperature. The reaction mixture was then further divided into two separate reactions (30 μl) for direct comparison of the extensions with and without apyrase. The extension mixture contained 20 U exonuclease-deficient (exo-) Klenow DNA polymerase (Amersham Pharmacia Biotech), Tris-acetate (pH 7.75), 0.5 mM EDTA, 5 mM Mg-acetate, 0.1% (w/v) bovine serum albumin (BioThema), 1 mM dithiothreitol and optionally 8 mU of apyrase (Sigma Chemical Co.) in a total volume of 70 μl. The extension mixture also contained nucleotides, dATP, dGTP, dTTP and dCTP (1.4 μM final concentration), where dCTP was labeled with Cy5 or Cy3 (Amersham Pharmacia Biotech). The reaction was carried out for 15 min at room temperature. The resulting extension products were washed twice with Tris-EDTA and the non-biotinylated extension products were isolated by adding 25 μl 0.1 M NaOH. The eluted strand was neutralized by 12.5 μl 0.2 M HCl and was printed on a glass slide by the GMS 417 Arrayer (Genetic MicroSystem, USA) and then was scanned using GMS 418 Scanner (Genetic MicroSystem, USA). The obtained results were analyzed using GenePix2.0 software (Axon Instruments, USA).

Apyrase Mediated Allele-specific Extension Using Fluorescent Labeled Nucleotide Evaluated in a Microarray Format

After amplification, the amplicons of wiaf 1764 were immobilized onto streptavidin coated beads as outlined above. The biotinylated inner primer was primer 3 and not primer 4 (Table 1) with the reason that for this assay the eluted strand is the extension substrate, thus to have the same match and mismatch configurations, the biotinylated inner primer was changed. The immobilized PCR product of wiaf 1764 was incubated in 20 μl 0.1 M NaOH for 5 min. The eluted strand was neutralized by 10 μl 0.2 M HCl and 4 μl annealing buffer (100 mM Tris-acetate pH 7.75, 20 mM Mg-acetate) was added. The eluted and neutralized single stranded DNA was divided into two parallel reactions in a microtiter-plate (17 μl/well) and extension primers (0.1 μM) were added. Hybridization was performed by incubation at 72° C. for 5 min and then cooling to room temperature. Here, the extension primers were modified to have amino groups at the 5′-end to allow covalent binding to pre-activated Silyated Slides (Cel Associates Inc, Texas, USA). In order to improve hybridization and extension the primers were extended with a 15-mer spacer (T₁₅) in the 5′-end (Table 1). One microliter of the primer-template hybrid was manually spotted on Silyated Slides (Cel Associates Inc) and the covalent coupling was performed in a humid chamber at 37° C. for 16 h. After coupling, 1 mU apyrase (1 μl) was added to each spot. A polymerization mixture was prepared and 4 μl was added to the spots immediately after addition of apyrase. The polymerization mixture contained Tris-acetate (pH 7.75), 0.5 mM EDTA, 5 mM Mg-acetate, 0.1% (w/v) bovine serum albumin (BioThema), 1 mM dithiothreitol, 0.08 μM (final concentration) dCTP labeled with Cy3 (Amersham Pharmacia Biotech) and 1 U exonuclease-deficient (exo-) Klenow DNA polymerase (Amersham Pharmacia Biotech). Polymerization was allowed to proceed for 15 min and the microarray slide was washed briefly with water and then scanned using GMS 418 Scanner (Genetic MicroSystem, USA). The data was analyzed by using GenePix2.0 software (Axon Instruments, USA).

Pyrosequencing

Single stranded DNA with annealed sequence primer (the substrate) (Table 1) were used for pyrosequencing. Real-time pyrosequencing was performed at 28° C. in a total volume of 50 μl in an automated 96-well PyroSequencer using PSQ™ SNP 96 enzymes and substrates (Pyrosequencing AB, Uppsala, Sweden).

Results

Two different approaches of apyrase mediated allele-specific extensions were investigated. The assays included a luminometric assay and two assays based on fluorescent labeled nucleotides. All the results obtained with these assays were confirmed by pyrosequencing. Four SNPs with the eight alternative 3′-end primer-template configurations were investigated (Table 1 and Table 2). The SNPs were codon 72 of the p53 gene (C or G), wiaf 1764 (G or T), nucleotide position 677 in the MTHFR gene (C or T) and nucleotide position 196 in the GPIIIa gene (G or A). Thus, using two alternative allele-specific primers for each SNP, the following mismatches were possible; G-G and C-C for codon 72 (p53 gene), A-G and C-T for wiaf 1764, A-C and G-T for polymorphic position on the MTHFR gene, T-G and C-A for the SNP on the GPIIIa gene (Table 2).

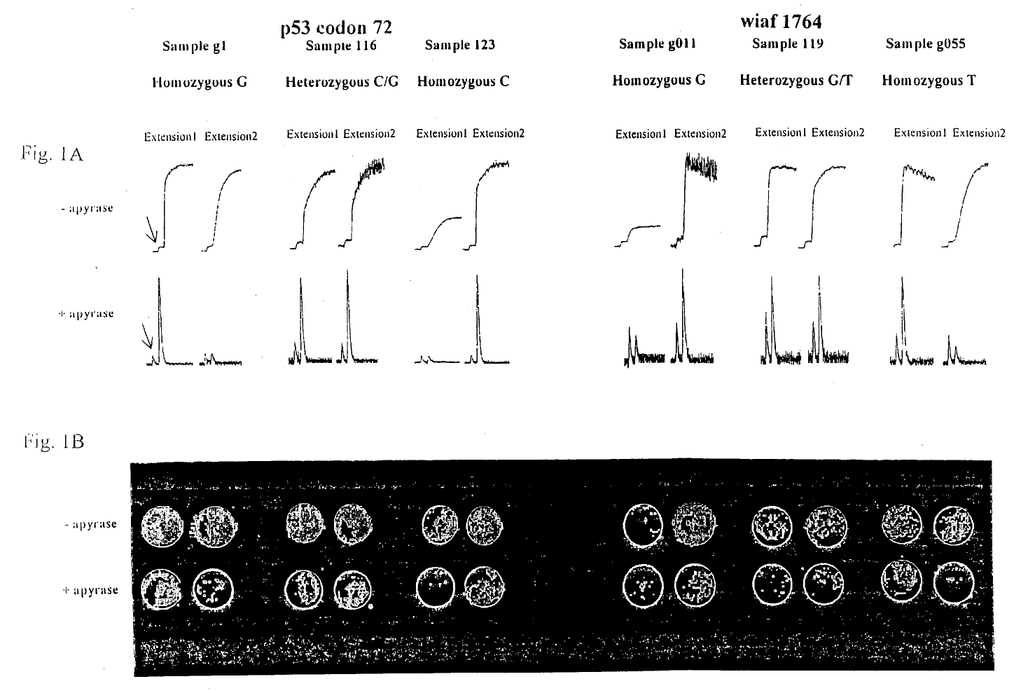

FIG. 1A shows the results of the luminometric assay with and without apyrase, for codon 72 of the p53 gene and wiaf 1764. When codon 72 is homozygous G (sample g1) (see Table 2), the mismatch signal is as high as the match signal but the slope of the curve indicates slower reaction kinetics (FIG. 1A top panel, extension 2). The same was observed for wiaf 1764 homozygous T (sample g055) (see Table 2). However, when apyrase was included in the allele-specific extension of these samples (FIG. 1A, lower panel) a dramatic difference was observed. The previous high signals for mismatch configurations, disappeared with the addition of apyrase. The extension ratios were calculated by taking the ratio of the high versus the low end-point signals. For example, the ratio for sample g1 in the p53 gene was 1.1 without apyrase and 13.9 with apyrase and for the sample g055 in wiaf 1764 the ratio was 1 without apyrase and 5 with apyrase (Table 2). This clearly shows that addition of apyrase affects the extension and simplifies interpretation. In these assays an extension ratio below or equal to 2 was interpreted as a sample being heterozygous. If the ratio was above or equal to 2.5 the SNP was scored as homozygous with the nucleotide at the 3′-end of the primer that produced the higher signal. Ratios between 2 and 2.5 were interpreted as uncertain. As shown in FIG. 1A and Table 2, in all cases with addition of apyrase the extension signals and extension ratios resulted in a correct genotype with the luminometric assay as compared to the pyrosequencing data. In fact, the extension ratios for all heterozygous SNPs were between 1.1 and 1.4 and the lowest extension ratio for a homozygous sample was 5. In contrast, without the inclusion of apyrase, five out of eight mismatches contributed with so high extension signals that the SNPs were wrongly scored (ratios in bold). The primer-template mismatches that were extended in these cases were G-G, C-T, G-T, T-G and C-A. FIG. 1B also shows the raw data of similar extensions on DNA immobilized on magnetic beads using fluorescent detection using a labeled nucleotide instead of a luminometric detection system. Thus, extension is performed by the same polymerase and the same primers, meaning that the discrimination behavior should be as in the luminometric assay. The results (extension ratios) are in good agreement with the luminometric assay (Table 2). Without apyrase the same mismatches gave high fluorescent signals leading to that the same homozygous samples were wrongly scored as heterozygous (ratios in bold in Table 2). However in presence of apyrase, no ambiguities or discordant results were observed (FIG. 1B lower panel and Table 2).

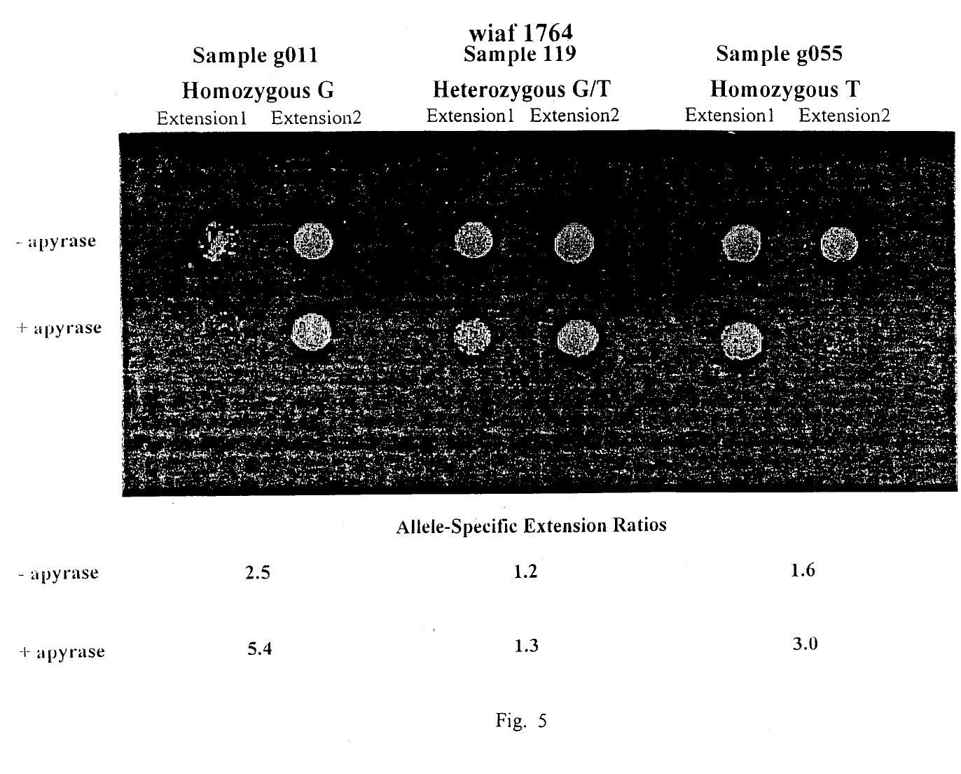

The foregoing example demonstrates that apyrase aids in the discrimination of mismatches in allele-specific extension. The extensions in these cases were performed on magnetic beads. The use of this technology in a microarray format was also evaluated. The SNP wiaf 1764 were amplified in 3 samples (g011, 119 and g055) and single stranded templates were obtained by immobilization of the PCR product onto magnetic beads. The eluted non-biotinylated strand was used in the subsequent extension experiments. Extension primers were modified to have a free amino group in the 5′-end as well as a 15 nucleotide long oligo dT spacer. The extension primers and the single strand target were hybridized, printed and then covalently coupled to the activated glass slides. The extension reaction mixtures were then added to microarray. FIG. 5 shows the raw-data of the analysis without and with apyrase. A direct comparison of the raw data obtained for samples of wiaf 1764 using both assays shows that the results of extension on chip are correct when apyrase is used while the same homozygous sample as in the other two assays (g055) has led to a high mismatch signal when apyrase was not used.

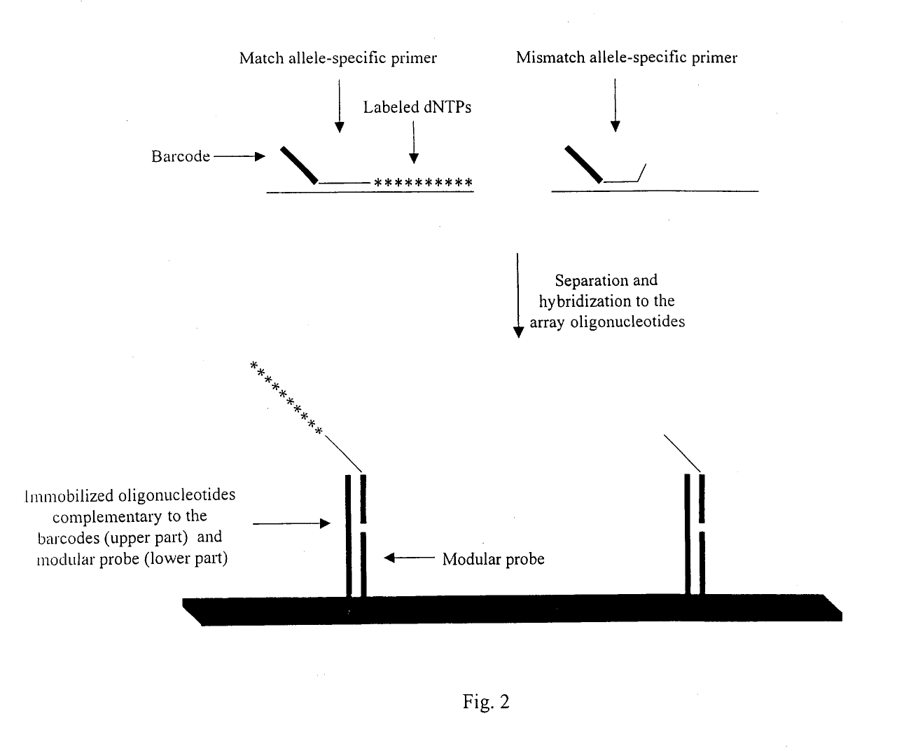

As shown above, a major advantage of using apyrase mediated allele specific extension (AMASE) is that the technique is applicable for high throughput genotyping. The present method may also be performed using barcodes (Fan et al. (2000) Genome Res. 10:853) as tags on the 5′-ends of allele-specific primers. In this way a multiplex AMASE of a set of SNPs is performed in a single tube (if the barcodes on the match and mismatch are different) or in two tubes (if the barcodes on the match and mismatch are identical). After performance of AMASE, the double-stranded AMASE products are heat separated and hybridized to barcode complementary oligonucleotides on the chip (FIG. 2). To improve the hybridization efficiency, at the hybridization step a modular probe is utilized (O'Meara et al. (1998) J. Clin. Microbiol. 36:2454). The modular probe can hybridize to its complementary segment of the immobilized oligonucleotide and improve the hybridization of barcode that is immediately downstream.

In conclusion, this example shows that single nucleotide polymorphisms can rapidly be scored with allele-specific primers in extension reactions. Furthermore, the present method can be used in a high-throughput format using microarrays.

TABLE 1

List of the primers.

| | | |
|---|---|---|
| SNP | Primer | 5′ 3′ |

| wiaf1764 | 1 | AGTGAAAACATTGAAAACACA |
|---|---|---|
| | 2 | AATGTTTTCACTGTCATAAAG |
| | 3 | TCCAATGTGTGAAAAATATATAC |
| | 4-Biotin | AGAACACATACGTTTTACCA |
| | Extension1 | ACTCCCTTCAGATCA |
| | Extension2 | ACTCCCTTCAGATCC |
| | Amino- | TTTTTTTTTTTTTTTATACAAC ACTC |
| | Extension1 | CCTTCAGATCA |
| | Amino- | TTTTTTTTTTTTTTTATACAAC ACTC |
| | Extension2 | CCTTCAGATCC |
| | Seq | CATTTGTTAAGCTTTT |
| p53 codon | 1 | ATGCTGTCCCCGGACGA |
| 72 | | |
| | 2 | CAGGAGGGGGCTGGTG |
| | 3-Biotin | TCCAGATGAAGCTCCCAG |
| | 4 | AGGGGCCGCCGGTGTA |
| | Extension1 | GCTGCTGGTGCAGGGGCCACGC |
| | Extension2 | GCTGCTGCTGCAGGGGCCACGG |
| | Seq | GCTGCTGGTGCAGGGGCCA |
| mthfr | 1 | CCTGACTGTCATCCCTATTGGCAG |
| | 2 | GGGACGATGGGGCAAGTGATG |
| | 3-Biotin | GCTGACCTGAAGCACTTGAAGGAG |
| | 4 | GCCTCAAAGAAAAGCTGCGTG |
| | Extension1 | GCTGCGTGATGATGAAATCGA |
| | Extension2 | GCTGCGTGATGATGAAATCGG |
| | Seq | AAGCTGCGTGATGATGAAA |
| | 1 | GCCATAGCTCTGATTGCTGGACTTC |
| gp3a | 2 | GCCTCACTCACTGGGAACTCGATG |
| | 3 | GCTGGACTTCTCTTTGGGCTCCTG |
| | 4-Biotin | ACAGTTATCCTTCAGCAGATTCTCCTT |
| | Extension1 | TCTTACAGGCCCTGCCTCC |
| | Extension2 | TCTTACAGGCCCTGCCTCT |
| | Seq | CCTGTCTTACAGGCCCTGCC |
| 1 and 2 = primers used in the outer PCR. 3 and 4 = primers used in the inner PCR. 1 and 3 are upstream primers and 2 and 4 are downstream primers. Biotin indicates the biotinylated primer in each set. Extension1 and Extension2 refer to the primers used as match and/or mismatch with the alternating base in the 3′-end. Ammo-Extension refer to the primers used on microarray. Seq = primers used for | | |
| # pyrosequenceing. Notice that when the Amino-Extension primers (for wiaf 1764) are used, the biotinylated inner primer was primer 3 and not primer 4 | | |

TABLE 2
Summary of extension results

| | | | Schematic | | Allele-Specific Extension Ratio | | | |
|---|---|---|---|---|---|---|---|---|
| | | | Representation of | | (high/how) | | | |
| | | Sequencing | Configurations | | Bioluminescence | | Fluorescent | |
| SNP | Sample | Result | Extension 1 | Extension 2 | − apyrase | + apyrase | − apyrase | + apyrase |
| p53 (G/C) | g1 | G/G | 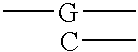 | 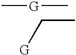 | 1.1 | 13.9 | 1.2 | 4.9 |
| | 116 | G/C | 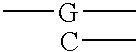 | 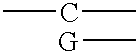 | 1.2 | 1.1 | 1.4 | 1.4 |
| | 123 | C/C | 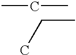 | 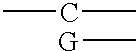 | 2.6 | 12 | 2.3 | 10.5 |
| wiaf 1764 (G/T) | g011 | G/G | 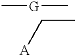 | 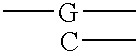 | 8.3 | 8 | 15.5 | 3.9 |
| | 119 | T/G | 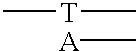 | 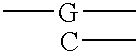 | 1.2 | 1.1 | 1.3 | 1.5 |
| | g055 | T/T | 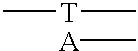 | 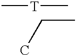 | 1 | 5 | 1.1 | 8.2 |
| MTHFR (C/T) | 1001 | C/C | 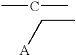 | 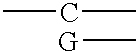 | 3.5 | 12 | 20 | 35 |
| | 1055 | T/C | 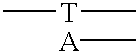 | 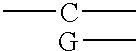 | 1 | 1.3 | 1.1 | 1.3 |
| | 1004 | T/T | 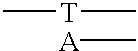 | 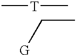 | 1.1 | 12 | 2.3 | 13 |
| GPIIIa (G/A) | 1267 | G/G | 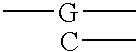 | 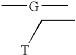 | 1.1 | 6.5 | 1.1 | 3.5 |
| | 1001 | G/A | 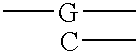 | 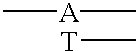 | 1.2 | 1.4 | 1.4 | 1.1 |
| | 1055 | A/A | 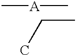 | 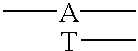 | 1.4 | 10 | 1.1 | 7.1 |

### EXAMPLE 2

### Apyrase Mediated Allele-specific Extension on DNA Microarrays

Apyrase mediated allele-specific extension (AMASE) for genotyping on DNA microarrays is described in this report. The method involves extension of the DNA samples in solution followed by hybridization to the DNA microarray as illustrated in FIG. 3.

Materials and Method

Microarray Preparation

Amino linked oligonucleotide capture probes suspended at a concentration of 20 μM in 3×SSC/0.01% sakrosyl were spotted using a GMS 418 arrayer (Affymetrix, USA) on silylated slides (CEL Associates, Houston, Tex.). Printed arrays were allowed to dry for 12 hours at room temperature followed by post processing to reduce unreacted aldehyde groups thereby minimising non-specific binding of target. Briefly the slides were washed twice in 0.2% SDS for 2 minutes, twice in dH₂O for 2 minutes and treated with sodium borohydride (0.75 g NaBH₄ dissolved in 225 ml PBS and 75 ml 100% ethanol) for 5 minutes. The arrays were then washed in 0.2% SDS three times for 1 minute, rinsed in H2O and dried by centrifugation for 1 minute at 500 g. Prior to use in hybridization, the arrays were prehybridized with buffer containing 5×Denharts solution, 6×SSC, 0.5% SDS and 0.1 μg/μl herring sperm DNA at 50° C. for 15 minutes followed by a brief rinse in dH₂O.

Oligonucleotides

The capture probes were synthesised with an amino group at the 5′ end to facilitate covalent immobilisation on the glass slide. A carbon spacer was also synthesised at the 3′ end to prevent any possible extension of the capture probe during hybridization, albeit unlikely due to the high salt conditions. The sequences of the capture probes and the allele specific extension primers are listed in Table 3.

DNA Preparation

PCR was carried out on human genomic DNA as previously described (Ahmadian et al. (2000) Anal. Biochem. 280:103)) to amplify 3 SNPs. The SNPs were codon 72 (C/G) in the p53 gene, nucleotide position 677 (C/T) in the methylenetetrahydrofolate reductase (MTHFR) gene and nucleotide position 196 (A/G) on the glycoprotein IIIa (GP3a) gene. To allow for immobilization of the PCR products on streptavidin beads and preparation of single strand DNA, biotinylated inner PCR primers were used. The biotinylated PCR-products (˜80 bp) were immobilised onto streptavidin-coated paramagnetic beads (Dynabeads® M-280, Dynal, Oslo, Norway) and by strand-specific elution a pure template for extension was obtained. Briefly, 100 μl of the PCR-products was captured by incubation for 15 minutes at room temperature with 5 mg/ml of beads in 100 μl binding/washing buffer (10 mM Tris-HCl (pH 7.5) 1 mM EDTA, 2 M NaCl, 1 mM β-mercaptoethanol, 0.1% Tween® 20). After washing and removal of supernatant, the strands were separated by incubation with 4 μl of 0.1 M NaOH for 5 minutes. The alkaline supernatant with the non-biotinylated strand was neutralised with 2.2 μl of 0.17 mM HCl and 1 μl of 100 mM Tris-Acetate pH 7.5, 20 mM MgAc₂.

Extension and Hybridization

Single Strand DNA:

The single strand DNA prepared above was divided into two aliquots and 2.5 pmoles of allele specific primers were annealed by incubation at 72° C. for 5 minutes in a volume of 20 μl. The annealed primer-DNA template was then further divided into two separate reactions for direct comparison of extension with and without apyrase. When multiplex extension was performed, single strand DNA from the 3 templates was mixed and allele specific primers were annealed.

Extension was performed in solution on 10 μl of the annealed DNA (corresponds to 25 ul of each PCR product) in a 60 μl volume containing 100 mM Tris-Acetate, 0.5 mM EDTA and 5 mM Mg-acetate with 1.4 μM pmoles of cy5 labelled dNTPs, 2.5 μg BSA, 1.25 mM DTT, 10 Units exonuclease-deficient (exo-) Klenow DNA polymerase and optionally 8 mU apyrase. Following incubation of the extension products at room temperature for 15 minutes, 60 μl 10×SSC/0.4% SDS was added to the extension reactions and 100 μl was then hybridized to the oligonucleotide microarray. Hybridization was performed on the GeneTAC hybridization station (Genomic solutions, MI, USA) at 50° C. for 20 minutes followed by washing in 2×SSC/0.1% SDS for 5 minutes proceeded by washing in 0.6×SSC for a further 5 minutes. The slides were briefly rinsed in H₂O and dried by centrifugation at 500 g for 1 minute.

Double Strand DNA:

One hundred and sixty microlitres of PCR product was treated with 16 Units of calf alkaline phosphatase and 32 Units exonuclease I at room temperature for 30 minutes. The enzymes were inactivated by incubation at 95° C. for 12 minutes and the DNA was divided into two aliquots and annealed to 5 pmoles of allele specific primers (incubation at 95° C. for 2 minutes followed by incubation at 72° C. for 5 minutes) in a volume of 100 μl. The annealed primer-DNA template was then further divided into two separate reactions for direct comparison of extension with and without apyrase. Extension was carried out on 50 μl of annealed double strand DNA (corresponds to 40 μl PCR product) in a 100 μl as described above. Following incubation at RT for 15 minutes, 25 μl 20×SSC and 12 μl 10% SDS was added to the extension mixture and 100 μl was hybridized to the slide as described above.

Data Analysis

The slides were scanned at optimal laser/PMT values using the GMS 417 scanner (Affymetrix, USA) and the features quantitated using GenePix 2.0 software (Axon Instruments, USA). Since different extension/hybridization experiments were compared, the micorarray data was subjected to a normalization procedure. This involved including a 66 mer oligonucleotide control and 18 mer extension probe (Table 3) together with the target DNA that was subjected to extension and hybridization. Since the intensity of this control should be constant from slide to slide it was used to normalize the slides for comparative purposes. Extension ratios were calculated by taking the ratio of the high versus the low signal.

Results

Results of AMASE for simultaneous genotyping of 3 SNPs on DNA microarrays are shown in Table 4. Inclusion of apyrase resulted in the correct genotype being called in all cases. However when apyrase was omitted, SNPs were incorrectly genotyped in 3 samples (illustrated in boldface type) if the criteria of a ratio ≧2.5 is required to call a homozygous genotype and ≦1.5 for a heterozygous sample. Two replicates of each feature were spotted which allows an estimation of the variability of the method and the standard deviation for each feature is shown in Table 4. The results in Table 4 are based on extension of single strand DNA while preliminary results for genotyping of double strand DNA gave ratios of 3.9±0.2 (with apyrase) versus 2.7±0.4 (without apyrase) for codon 72 of the p53 gene (sample g1).

TABLE 3

Sequence of capture and extension probes

| | | |
|---|---|---|
| SNP | Probe | Sequence (5′-3′) |

| | | |
|---|---|---|
| | Capture | TGA AGC TCC CAG AAT GCC |
| P53 | probe | |
| | Extension | AGA GGC TGC TCC CCC |
| | 1 | |
| | Extension | AGA GGC TGC TCC CCG |
| | 2 | |
| MTHFR | Capture | CAG CCT CAA AGA AAA GCT |
| | probe | |
| | Extension | GCG TGA TGA TGA AAT CGG |
| | 1 | |
| | Extension | GCG TGA TGA TGA AAT CGA |
| | 2 | |
| GPIIIa | Capture | CTT CTC TTT GGG CTC CTG |
| | probe | |
| | Extension | TCT TAC AGG CCC TGC CTC C |
| | 1 | |
| | Extension | TCT TAC AGG CCC TGC CTC T |
| | 2 | |
| Control | Capture | GGT GCA CGG TCT ACG AGA |
| | probe | |
| | Extension | CCT CCC GGG GCA CTC GCA |
| | probe | |
| | Extension | AGG CCT TGT GGT ACT GCC TGG TAG |
| | template | GGT GCT TGC GAG TGC CCC GGG AGG |
| | | TCT CGT AGA CCG TGC ACC |

TABLE 4
Genotyping of SNPs on microarrays using AMASE

| | Allele-Specific Extension |
|---|---|
| | Ratio (high/low) |
| Sequencing | Microarray |

| SNP | Sample | Result | − apyrase | + apyrase |
|---|---|---|---|---|
| p53 | g1 | G/G | 1.9 ± 0.2* | 8.8 ± 1.6* |
| (G/C) | | | | |
| | 116 | G/C | 2.5 ± 0.5* | 1.1 ± 0.1* |
| | 123 | C/C | 22.1 ± 0.5 | 37.9 ± 3.8 |
| THFR | 1001 | C/C | 19.8 ± 1.8 | 21.8 ± 2.9 |
| (C/T) | | | | |
| | 1055 | T/C | nd | nd |
| | 1004 | T/T | 21.3 ± 3.7 | 42 ± 5.1 |
| GPIIIa | 1267 | G/G | 2.2 ± 0.2 | 6.4 ± 1.6 |
| (G/A) | | | | |
| | 1001 | G/A | nd | nd |
| | 1055 | A/A | 8.6 ± 0.7 | 28.3 ± 3.9 |

*indicates that these samples were not genotyped in a multiplex format nd (not determined) indicates that these samples have yet to be tested

### EXAMPLE 3

### Double Stranded DNA Analysis of a SNP

Materials and Methods

PCR amplification of wiaf 1764 was performed as described in the foregoing examples. In Order to perform double-strand DNA analysis of this SNP (without strand separation by the use of beads), the excess of primers, nucleotides and the released PPi in the PCR had to be removed. For this purpose, the enzymes shrimp alkaline phosphatase (4 U) (Roche Diagnostics) and E. coli exonuclease I (8 U) (Amersham Pharmacia Biotech, Uppsala, Sweden) were added to 40 μl of each PCR product. Shrimp alkaline phosphatase was used to degrade PPi and the dNTPs while exonuclease I removed single-stranded DNA molecules including PCR primers. The enzymatic degradation was allowed to proceed for 30 min at room temperature. The mixtures were then heated to 97° C. for 12 min to deactivate the enzymes. The samples were divided into two tubes (20 μl in each) and pairs of allele-specific primer (0.25 μM) (one complementary to the forward strand and one complementary to the reverse strand) were added into each tube. The sequence of primers was A-forward TACAACACTCCCTTCAGATCA, A-reverse TACCATTTGTTAAGCTTTTGT, C-forward TACA ACACTCCCTTCAGATCC and C-reverse ACCATTTGTTAAGCTTTTGG. The primer pairs in different tubes differed only in their 3′-ends (underlined bases indicate the alternating 3′-ends) to allow discrimination by allele-specific extension using DNA polymerase. After addition of primers, the samples were incubated at 97° C. for 2 min and then cooled to room temperature, allowing hybridization of allele-specific primer pairs. The content of each well was then further divided into two separate reactions for comparison of extension analysis with and without apyrase. Extension and real-time luminometric monitoring was performed at 25° C. in a Luc96 pyrosequencer instrument (Pyrosequencing, Uppsala, Sweden). An extension reaction mixture was added to the samples (10 μl) with annealed primers (the substrate) to a final volume of 50 μl. The extension reaction mixture contained 10 U exonuclease-deficient (exo-) Klenow DNA polymerase (Amersham Pharmacia Biotech, Uppsala, Sweden), 4 μg purified luciferase/ml (BioThema, Dalarö, Sweden), 15 mU recombinant ATP sulfurylase, 0.1 M Tris-acetate (pH 7.75), 0.5 mM EDTA, 5 mM Mg-acetate, 0.1% (w/v) bovine serum albumin (BioThema), 1 mM dithiothreitol, 10 μM adenosine 5′-phosphosulfate (APS), 0.4 mg polyvinylpyrrolidone/ml (360 000), 100 μg D-luciferin/ml (BioThema) and 8 mU of apyrase (Sigma Chemical Co., St. Louis, Mo., USA) when applicable. Prior to nucleotide addition and measuring of emitted light, pyrophosphate (PPi) was added to the reaction mixture (0.08 μM). The PPi served as a positive control for the reaction mixture as well as peak calibration. All the four nucleotides (Amersham Pharmacia Biotech, Uppsala, Sweden) were mixed and were dispensed to the extension mixture (0.8 μM, final concentration). The emitted light was detected in real-time.

Results and Discussion

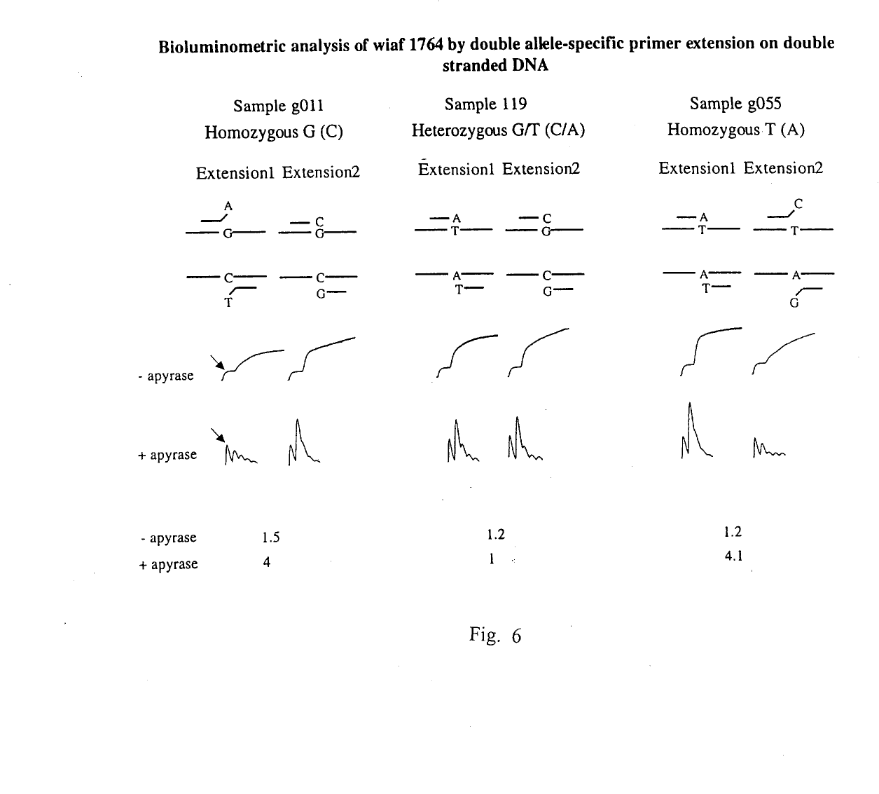

FIG. 6 shows the results of bioluminometric analysis of wiaf 1764. The SNP wiaf 1764 has the variants G and/or T (C and/or A). Two pairs of allele-specific primers were used to analyze this SNP, one complementary to the forward strand and one complementary to the reverse strand. The primer pairs differed in their 3′-ends to allow discrimination by allele-specific extension using DNA polymerase, (extension 1 and extension 2 in FIG. 6). Prior to hybridization of allele-specific primers the PCR products were treated by shrimp alkaline phosphatase and exonuclease I to remove the excess of primers, nucleotides and PPi. This allowed heat separation of double-stranded DNA and direct analysis of the PCR product without strand separation by using beads. Thus, allele-specific extension was performed on both strands of a double-stranded DNA. The bioluminimetric assay was performed without using apyrase (−apyrase in FIG. 6) and by the use of apyrase (+apyrase in FIG. 6). All three variants of wiaf 1764 were analyzed and the ratios between the match and mismatch signals were calculated. The ratios are outlined in the bottom of FIG. 6. As it is shown all variants of the SNP could correctly be scored when apyrase was used in the system (ratios 4, 1 and 4.1 for samples g011, 119 and g055 respectively) while two of the same samples were wrongly scored when apyrase was not applied (ratios 1.5 and 1.2 for samples g011 and g055 respectively).

The advantage of using two primers in AMASE is that these can be utilized in a PCR amplification assay when a thermostable nucleotide-degrading enzyme (e.g. alkaline phosphatase) is available. A perfectly match primer pair will give rise to amplification while mismatch primer pair will not. After amplification, the PCR products are directly analyzed by a luminometric assay since the same primer pair is used in allele-specific extension. Another advantage of using two allele-specific primers is that the sensitivity increases by a factor of two because signals of two extensions are obtained instead of one.

### EXAMPLE 4

### Introduced Mismatch in Allele-specific Primers to Improve Allele-specific Extension

The present example describes a method of allele-specific extension using apyrase and an introduced mismatch in the allele-specific primer, with non-stringent conditions for extension.

PCR amplification of GPIIIa gene was performed as described in the foregoing examples. The SNP in the GPIIIa gene has the variants C/T (G/A). The PCR products were immobilized on magnetic beads and single-stranded DNA was obtained by alkaline treatment as described in the foregoing examples. The immobilized single-stranded DNA was used as target template in the assay. Two different primers were designed. The sequence of primers was CTG TCT TAC AGG CCC TGC CTG CG for GPIIIC and CTG TCT TAC AGG CCC TGC CTG TG for GPIIIT. The immobilized strand was resuspended in 32 μl H₂O and 4 μl annealing buffer (100 mM Tris-acetate pH 7.75, 20 mM Mg-acetate). The single stranded DNA was divided into two parallel reactions in a microtiter-plate (18 μl/well) and 0.2 μM (final concentration) of primers were added to the single stranded templates in a total volume of 20 μl. The primers were designed so that the 3′-end was one base after the SNP site and was complementary to the target DNA (indicated in italics). Thus, the base before the 3′-end (3′-1) was complementary to the SNP variants and was the only difference between the two allele-specific primers (underlined bases). The base before the SNP site (3′-2) on both primers (indicated in bold) was an introduced mismatch to the target DNA (G on the primers and G on the target DNA). Therefore, when the allele-specific base in the primer does not match to the target template, two mismatches (positions 3′-1 and 3′-2) and one match (position 3′) will be made between the template and the last 3 bases in the primer (FIG. 7). When the allele-specific base in the primer does match to the target template, the last two bases in the 3′-end of the primer (positions 3′and 3′-1) will be complementary to the target DNA while the introduced mismatch (position 3′-2) is not complementary. Raw-data were obtained from a luminometric assay. The conditions (enzymes, substrates etc) of performance of the luminometric assay are as described in the foregoing examples. Prior to nucleotide addition and measurement of emitted light, pyrophosphate (PPi) was added to the reaction mixture (0.02 μM). The PPi served as a positive control for the reaction mixture as well as peak calibration. All three variants of the SNP were investigated with (+apyrase) and without (−apyrase) addition of apyrase (FIG. 7). As shown in FIG. 7, Sample 1055 is homozygous A in the target DNA template. Thus, an allele-specific primer containing the base C at 3′-1 position is a mismatch to the target (C to A mismatch) and since the 3′-2 position is also a mismatch (G to G mismatch), the two mismatches disrupt the hydrogen bind of the match base at the 3′ position of the primer (G to C match) and no extension is observed. When the allele-specific primer contains a matching base to the target DNA at the 3′-1 position (T to A match), two complementary bases are made (at 3′-1 and 3′ position) between the primer and template. In a non-stringent extension condition (extension at 25° C.), the mismatch base at position 3′-2 does not remove the two complementary bases at 3′-1 and 3′, which leads to extension. The same explanation can be used for sample 1267 that is homozygous G. In the heterozygous case (sample 1001) both allele-specific bases are complementary to the respective variant and give rise to extension signals.

We claim:

1. A method for detecting an allele-specific base at a predetermined position in a target nucleic acid molecule comprising providing a first and a second hybridization mixture, said first hybridization mixture comprising said target nucleic acid molecule, and a primer that hybridizes to a region of said target nucleic acid molecule and that has a 3′-terminus that is complementary to a non-mutated base at said predetermined position, said second hybridization mixture comprising said target nucleic acid molecule, and a primer that hybridizes to a region of said target nucleic acid molecule and that has a 3′-terminus that is complementary to a mutated base at said predetermined position; adding a primer extension reaction mixture to each of said first and second hybridization mixtures, said primer extension reaction mixture comprising a DNA polymerase, nucleotides, and a nucleotide degrading enzyme; and determining primer extension efficiency in each of said first and second mixtures, whereby greater efficiency in the mixture comprising the primer having a 3′-terminus that is complementary to the mutated base is indicative of the presence of the mutated base in the target nucleic acid, and whereby greater efficiency in the mixture comprising the primer having a 3′-terminus that is complementary to the non-mutated base is indicative of the presence of the non-mutated base in the target nucleic acid.

2. The method of claim 1 wherein the nucleotide degrading enzyme is apyrase.

3. The method of claim 1 wherein the target nucleic acid is amplified before hybridization.

4. The method of claim 1 wherein the target nucleic acid is immobilized.

5. The method of claim 1 wherein primer extension efficiency is measured by an assay selected from mass spectroscopy, a luminometric assay, a fluorescent assay, and pyrosequencing.

6. The method of claim 1 wherein said method is performed in a solid phase microarray format.

7. The method of claim 1 wherein said primers are tagged on the 5′-ends by barcodes.

8. The method of claim 1 wherein the target nucleic acid is double stranded.

9. A method for detecting an allele-specific base at a predetermined position in a target nucleic acid comprising conducting a first and a second allele-specific primer extension reaction using a first and a second primer, respectively that hybridizes to a region of said target nucleic acid, each of said primers having: 1) a 3′-end base that is complementary to the base that is 5′ of the predetermined position in the target; 2) a base one position from the 3′-end that in the first primer is complementary to a non-mutated based at the predetermined position, and in the second primer is complementary to a mutated base at the predetermined position; and 3) a base two positions from the 3′-end that is the same as the base that is 3′ of the predetermined position in the target; and determining primer extension efficiency in said first and second reactions, whereby greater efficiency in said first reaction is indicative of the presence of a non-mutated base at said predetermined position, and whereby greater efficiency in said second reaction is indicative of the presence of a mutated base at said predetermined position.

10. The method of claim 9 wherein said extension reactions are performed in the presence of a nucleotide degrading enzyme.

11. The method of claim 10 wherein said nucleotide degrading enzyme is apyrase.

12. The method of claim 9 wherein said target nucleic acid is amplified before hybridization.

13. The method of claim 9 wherein said target nucleic acid is immobilized.

14. The method of claim 9 wherein said primer extension efficiency is measured by an assay selected from mass spectroscopy, a luminometric assay, a fluorescent assay, and pyrosequencing.

15. The method of claim 9 wherein said method is performed in a solid phase microarray format.

16. The method of claim 9 wherein said primers are tagged at the 5′-ends by barcodes.

17. The method of claim 9 wherein said target nucleic acid is double stranded.
